(19) United States
(12) Patent Application Publication
(10) Pub. No.: US 2018/0338690 A1
(43) Pub. Date: Nov. 29, 2018

(54) MEASURING DEVICE INCLUDING LIGHT SOURCE, PHOTODETECTOR, AND CONTROL CIRCUIT

(71) Applicant: Panasonic Intellectual Property Management Co., Ltd., Osaka (JP)

(72) Inventors: TERUHIRO SHIONO, Osaka (JP); TAKAMASA ANDO, Osaka (JP)

(21) Appl. No.: 15/961,414

(22) Filed: Apr. 24, 2018

(30) Foreign Application Priority Data
May 23, 2017 (JP) 2017-101837

(51) Int. Cl. A61B 5/026; G01N 33/49; G01F 1/66; A61B 5/00

(52) U.S. Cl. CPC A61B 5/0261; G01N 33/49; G01F 1/661; A61B 2576/00; A61B 2562/0233; A61B 2562/046; A61B 5/0042

(57) ABSTRACT

A measuring device includes a light source that emits at least one first light pulse and at least one second light pulse toward a target part of an object, a photodetector that detects at least one first reflected light pulse returning from the target part and at least one second reflected light pulse returning from the target part, and a control circuit that controls the light source and the photodetector. The control circuit causes the light source to emit the at least one first light pulse and the at least one second light pulse at different timings. The control circuit causes the photodetector to detect a first component and output a first electric signal representing the first component. The control circuit causes the photodetector to detect a second component and output a second electric signal representing the second component.

### BACKGROUND

### 1. Technical Field

The present disclosure relates to a measuring device.

### 2. Description of the Related Art

As basic parameters for determining the health condition of a human, heart rate, blood flow volume, blood pressure, oxygen saturation, and the like are widely used.

For the acquisition of biological information, electromagnetic waves falling within a wavelength range of near infrared radiation, i.e. approximately 700 nm to approximately 2500 nm, are frequently used. Among them, near infrared rays of comparatively short wavelengths, e.g. approximately not longer than 950 nm, are especially frequently used. Such near infrared rays of short wavelengths have the property of being transmitted through body tissue such as muscles, fat, bones, and the like at comparatively high transmittances. Meanwhile, such near infrared rays have the property of being easily absorbed into oxyhemoglobin (HbO₂) and deoxyhemoglobin (Hb) in the blood. As a biological information measuring method that involves the use of these properties, near infrared spectroscopy (hereinafter abbreviated as “NIRS”) is known. Use of NIRS makes it possible to measure, for example, the amount of change in blood flow in the brain or the amounts of change in oxyhemoglobin concentration and deoxyhemoglobin concentration in the blood. It is also possible to estimate the state of activity of the brain on the basis of the amount of change in blood flow, the oxygen state of hemoglobin, or the like.

Japanese Unexamined Patent Application Publication No. 2007-260123 and Japanese Unexamined Patent Application Publication No. 2003-337102 disclose devices based on NIRS.

### SUMMARY

In one general aspect, the techniques disclosed here feature a measuring device including: a light source that emits at least one first light pulse and at least one second light pulse toward a target part of an object, the at least one second light pulse being different in light power from the at least one first light pulse; a photodetector that detects at least one first reflected light pulse returning from the target part and at least one second reflected light pulse returning from the target part; and a control circuit that controls the light source and the photodetector. The control circuit causes the light source to emit the at least one first light pulse and the at least one second light pulse at different timings. The control circuit causes the photodetector to detect a first component and output a first electric signal representing the first component. The first component is a component of light included in the at least one first reflected light pulse. The control circuit causes the photodetector to detect a second component and output a second electric signal representing the second component. The second component is a component of light included in the at least one second reflected light pulse during a falling period. The falling period is a period from a point in time at which the at least one second reflected light pulse starts decreasing in light power to a point in time at which the at least one second reflected light pulse finishes decreasing in light power.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1A is a schematic view for explaining a configuration of a biological measuring device according to Embodiment 1 of the present disclosure and the way in which a biological measurement is carried out;

FIG. 1B is a diagram schematically showing an internal configuration of a photodetector according to Embodiment 1 of the present disclosure and the flow of signals;

FIG. 2A is a diagram showing a time distribution of a single light pulse that is emitted light;

FIG. 2B is a diagram showing time distributions of a total light power (solid line) in a stationary state and a power (dashed line) of light having passed through a region of change in brain blood flow;

FIG. 2C is a diagram showing time distributions, during a falling period, of the total light power (solid line) in the stationary state and the power (dashed line) of the light having passed through the region of change in brain blood flow;

FIG. 2D is a diagram showing time distributions of the total light power (solid line) in the stationary state, the power (dashed line) of the light having passed through the region of change in brain blood flow, and a degree of modulation (chain line);

FIG. 3 is a diagram schematically showing a time distribution (upper row) of first and second light pulses, a time distribution (middle row) of a light power that is detected by the photodetector in a case where the first and second light pulses are emitted, and the timing and charge storage (lower row) of an electronic shutter according to Embodiment 1 of the present disclosure;

FIG. 4A is a front view showing changes in blood flow that are present in the surface and interior of a target part;

FIG. 4B is a side cross-sectional view showing the changes in blood flow that are present in the surface and interior of the target part;

FIG. 5A is a diagram schematically showing changes in blood flow in the surface of the target part as detected by the first light pulse;

FIG. 5B is a diagram schematically showing changes in blood flow in the surface and interior of the target part as detected by the second light pulse;

FIG. 5C is a diagram schematically showing changes in blood flow in the interior of the target part as derived by image computations;

FIG. 5D is a diagram schematically showing changes in blood flow in the interior of the target part as derived by further image computations;

FIG. 6 is a diagram schematically showing a time distribution (upper row) of first and second light pulses, a time distribution (middle row) of a light power on the photodetector in a case where the first and second light pulses are emitted, and the timing and charge storage (lower row) of the electronic shutter according to Modification 1 of Embodiment 1 of the present disclosure;

FIG. 7 is a diagram schematically showing a time distribution (upper row) of first and second light pulses, a time distribution (middle row) of a light power on the photodetector in a case where the first and second light pulses are emitted, and the timing and charge storage (lower row) of the electronic shutter according to Modification 2 of Embodiment 1 of the present disclosure;

FIG. 8 is a diagram schematically showing a time distribution (upper row) of first and second light pulses, a time distribution (middle row) of a light power on the photodetector in a case where the first and second light pulses are emitted, and the timing and charge storage (lower row) of the electronic shutter according to Modification 3 of Embodiment 1 of the present disclosure;

FIG. 9A is a diagram schematically showing a time distribution (upper row) of first and second light pulses, a time distribution (middle row) of a light power on the photodetector in a case where the first and second light pulses are emitted, and the timing and charge storage (lower row) of an electronic shutter according to Embodiment 2 of the present disclosure;

FIG. 9B is a diagram schematically showing an internal configuration of the photodetector according to Embodiment 2 of the present disclosure and the flow of electric signals and control signals;

FIG. 10A is a schematic view for explaining a configuration of a biological measuring device according to Embodiment 3 of the present disclosure and the way in which a biological measurement is carried out;

FIG. 10B is a diagram schematically showing an internal configuration of a photodetector according to Embodiment 3 of the present disclosure and the flow of electric signals and control signals;

FIG. 11 is a diagram schematically showing a time distribution (upper row) of first and second light pulses, a time distribution (middle row) of a light power on the photodetector in a case where the first and second light pulses are emitted, and the timing and charge storage (lower row) of an electronic shutter according to Embodiment 3 of the present disclosure;

FIG. 12 is a diagram schematically showing a time distribution (upper row) of first and second light pulses, a time distribution (middle row) of a light power on the photodetector in a case where the first and second light pulses are emitted, and the timing and charge storage (lower row) of the electronic shutter according to Modification 1 of Embodiment 3 of the present disclosure; and

FIG. 13 is a diagram schematically showing a time distribution (upper row) of first and second light pulses, a time distribution (middle row) of a light power on the photodetector in a case where the first and second light pulses are emitted, and the timing and charge storage (lower row) of the electronic shutter according to Modification 2 of Embodiment 3 of the present disclosure.

### DETAILED DESCRIPTION

Prior to a description of embodiments of the present disclosure, underlying knowledge forming basis of the present disclosure is described.

Japanese Unexamined Patent Application Publication No. 2007-260123 discloses an endoscopic device based on NIRS. The endoscopic device disclosed in Japanese Unexamined Patent Application Publication No. 2007-260123 uses light pulses as illuminating light to observe blood flow information in blood vessels buried in body tissue covered with visceral fat. In so doing, by making an imaging timing later than the timing of incidence of a light pulse, imaging of intense noise light that returns temporally early is avoided. This improves the S/N ratio of signal light returning from a deep place in the body tissue.

Japanese Unexamined Patent Application Publication No. 2003-337102 discloses a biological activity measuring device based on NIRS. This measuring device includes a light source section that generates infrared light, a photodetection section that detects infrared light from a target part of a living body, and a controller. This measuring device measures brain functions in a non-contact manner.

The device disclosed in Japanese Unexamined Patent Application Publication No. 2003-337102 makes it possible to measure brain activity by means of NIRS. However, since light reflected by the target part includes intense noise light that returns temporally early, the S/N ratio of a signal that is detected is undesirably low.

It is conceivable that this problem may be solved by combining the technology of Japanese Unexamined Patent Application Publication No. 2007-260123 with the device of Japanese Unexamined Patent Application Publication No. 2003-337102. That is, it is conceivable that the influence of intense noise light that returns temporally early can be curbed by making the timing of detection of light later than the timing of incidence of a light pulse.

However, the inventors studied and found that, even with such measures being taken, it is difficult to make the S/N ratio sufficiently high. Emitted light having entered the brain scatteringly propagates through the brain. Detection of the light makes it possible to acquire information on blood flow in the brain. However, on the optical path from inside the brain to the device, i.e. on a return path, the light always passes through a region of distribution of blood flow near the surface of the living body, i.e. scalp blood flow. Therefore, information on scalp blood flow, as well as the information on brain blood flow, is greatly superimposed onto the light. As a result of that, accurate information on brain blood flow cannot be obtained simply by detecting the returning light. That is, with a method based on the combination of the conventional technologies, it is impossible to make the S/N ratio of a detection signal sufficiently high.

The inventors have found the foregoing problems and conceived of a novel measuring device.

The present disclosure encompasses measuring devices according to the following items.

### Item 1

A measuring device according to Item 1 of the present disclosure includes: a light source that emits at least one first light pulse and at least one second light pulse toward a target part of an object, the at least one second light pulse being different in light power from the at least one first light pulse; a photodetector that detects at least one first reflected light pulse returning from the target part and at least one second reflected light pulse returning from the target part; and a control circuit that controls the light source and the photodetector. The control circuit causes the light source to emit the at least one first light pulse and the at least one second light pulse at different timings. The control circuit causes the photodetector to detect a first component and output a first electric signal representing the first component. The first component is a component of light included in the at least one first reflected light pulse. The control circuit causes the photodetector to detect a second component and output a second electric signal representing the second component. The second component is a component of light included in the at least one second reflected light pulse during a falling period. The falling period is a period from a point in time at which the at least one second reflected light pulse starts decreasing in light power to a point in time at which the at least one second reflected light pulse finishes decreasing in light power.

### Item 2

In the measuring device according to Item 1, the object may be a living body, and

the measuring device according to Item 1 may further include a signal processing circuit that generates blood flow information on the target part through computation based on the first electric signal and the second electric signal.

### Item 3

In the measuring device according to Item 2, the first electric signal may include blood flow information on a surface of the target part,

the second electric signal may include blood flow information on the surface and an interior of the target part, and

the signal processing circuit may generate blood flow information on the interior of the target part.

### Item 4

In the measuring device according to Item 2 or 3, the photodetector may be an image sensor including photodetection cells arrayed two-dimensionally, and

each of the photodetection cells may accumulate the first component as a first signal charge, accumulate the second component as a second signal charge, output, as the first electric signal, an electric signal representing a total amount of the first signal charge, and output, as the second electric signal, an electric signal representing a total amount of the second signal charge.

### Item 5

In the measuring device according to Item 4, the control circuit may cause the image sensor to output a first image signal representing a first two-dimensional distribution of the total amount of the first signal charge accumulated in each of the photodetection cells during a first period, a second image signal representing a second two-dimensional distribution of the total amount of the second signal charge accumulated in each of the photodetection cells during a second period that is identical to or different from the first period, a third image signal representing a third two-dimensional distribution of the total amount of the first signal charge accumulated in each of the photodetection cells during a third period preceding the first period, and a fourth image signal representing a fourth two-dimensional distribution of the total amount of the second signal charge accumulated in each of the photodetection cells during a fourth period preceding the second period, and

the signal processing circuit may receive the first to fourth image signals from the image sensor, generate a first difference image representing a difference between the first image signal and the third image signal, and generate a second difference image representing a difference between the second image signal and the fourth image signal.

### Item 6

In the measuring device according to Item 5, 0.1≤M₁/M₂≤10 may be satisfied when

the first difference image includes first pixels each of which has a pixel value exceeding a first threshold, the first pixels forming a first region,

the second difference image includes second pixels each of which has a pixel value exceeding a second threshold, the second pixels forming a second region,

M₁ is an average pixel value of first pixels included in a part of the first region that overlaps the second region, and

M₂ is an average pixel value of second pixels included in a part of the second region that overlaps the first region.

### Item 7

In the measuring device according to any of Items 4 to 6, the at least one first light pulse may have a pulse width that is shorter than a length of time that the photodetector accumulates the first signal charge.

### Item 8

In the measuring device according to any of Items 4 to 6, the at least one first light pulse may have a pulse width that is longer than a length of time that the photodetector accumulates the first signal charge.

### Item 9

In the measuring device according to any of Items 4 to 8, the at least one first light pulse may comprise a plurality of first light pulses,

the at least one second light pulse may comprise a plurality of second light pulses,

the control circuit may cause the light source to repeatedly emit the plurality of first light pulses during a first frame period,

the control circuit may cause the photodetector to accumulate the first signal charge in synchronization with emission of each of the plurality of first light pulses,

the control circuit may cause the light source to repeatedly emit the plurality of second light pulses during a second frame period that follows the first frame period, and

the control circuit may cause the photodetector to accumulate the second signal charge in synchronization with emission of each of the plurality of second light pulses.

### Item 10

In the measuring device according to any of Items 1 to 8, the at least one first light pulse may comprise a plurality of first light pulses,

the at least one second light pulse may comprise a plurality of second light pulses,

the control circuit may cause the light source to alternately emit each of the plurality of first light pulses and each of the plurality of second light pulses, and

a time interval from a center of each of the plurality of first light pulses to a center of a second light pulse that is emitted immediately after each of the plurality of first light pulses may be shorter than a time interval from a center of each of the plurality of second light pulses to a center of a first light pulse that is emitted immediately after each of the plurality of second light pulses.

### Item 11

In the measuring device according to any of Items 1 to 10, either of the at least one first light pulse and the at least one second light pulse may have a wavelength of not shorter than 650 nm and shorter than 805 nm and the other of the at least one first light pulse and the at least one second light pulse may have a wavelength of longer than 805 nm and not longer than 950 nm.

### Item 12

In the measuring device according to any of Items 1 to 11, the at least one second light pulse may be higher in light power than the at least one first light pulse.

### Item 13

A measuring device according to Item 13 of the present disclosure includes: a light source that emits first light pulses and second light pulses toward a target part of an object; a photodetector that detects first reflected light pulses returning from the target part and second reflected light pulses returning from the target part; and a control circuit that controls the light source and the photodetector. Light power of each of the second light pulses is higher than light power of each of the first light pulses. The control circuit causes the light source to alternately emit each of the first light pulses and each of the second light pulses. The control circuit causes the photodetector to detect a component of light included in the first reflected light pulses. The control circuit causes the photodetector to detect a component of light included in the second reflected light pulses.

In the present disclosure, all or some of the circuits, units, devices, members, or sections or all or some of the functional blocks in the block diagrams may be implemented as one or more of electronic circuits including, but not limited to, a semiconductor device, a semiconductor integrated circuit (IC), or an LSI (large scale integration). The LSI or IC can be integrated into one chip, or also can be a combination of multiple chips. For example, functional blocks other than a memory may be integrated into one chip. The name used here is LSI or IC, but it may also be called system LSI, VLSI (very large scale integration), or ULSI (ultra large scale integration) depending on the degree of integration. A Field Programmable Gate Array (FPGA) that can be programmed after manufacturing an LSI or a reconfigurable logic device that allows reconfiguration of the connection or setup of circuit cells inside the LSI can be used for the same purpose.

Further, it is also possible that all or some of the functions or operations of the circuits, units, devices, members, or sections are implemented by executing software. In such a case, the software is recorded on one or more non-transitory recording media such as a ROM, an optical disk, or a hard disk drive, and when the software is executed by a processor, the software causes the processor together with peripheral devices to execute the functions specified in the software. A system or device may include such one or more non-transitory recording media on which the software is recorded and a processor together with necessary hardware devices such as an interface.

In the following, embodiments of the present disclosure are more specifically described. Note, however, that an unnecessarily detailed description may be omitted. For example, a detailed description of a matter that has already been well known and a repeated description of substantially identical configurations may be omitted. This is intended to prevent the following description from becoming unnecessarily redundant and facilitate understanding of persons skilled in the art. It should be noted that the inventors provide the accompanying drawings and the following description so that persons skilled in the art can sufficiently understand the present disclosure, and do not intend to thereby limit the subject matters recited in the claims. In the following description, identical or similar constituent elements are given the same reference signs.

In the following, embodiments are described with reference to the drawings.

### Embodiment 1

First, a biological measuring device according to Embodiment 1 of the present disclosure is described.

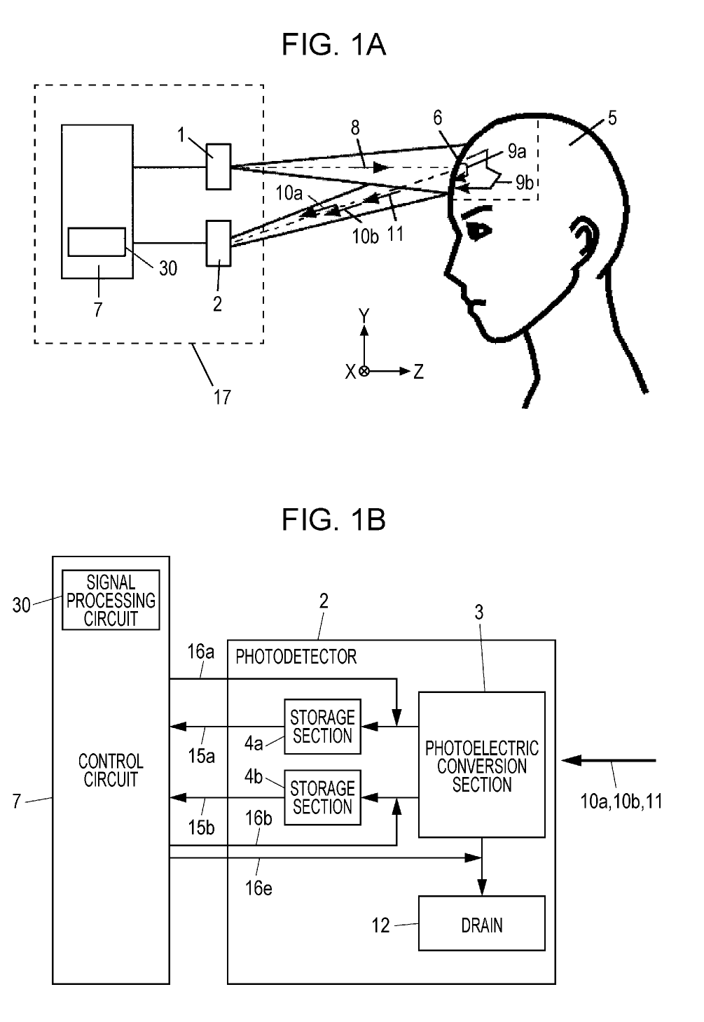

FIG. 1A is a schematic view for explaining a configuration of a biological measuring device 17 according to Embodiment 1 of the present disclosure and the way in which a biological measurement is carried out. FIG. 1B is a diagram schematically showing an internal configuration of a photodetector 2 according to Embodiment 1 of the present disclosure and the flow of signals.

The biological measuring device 17 according to Embodiment 1 includes a light source 1, the photodetector 2, and a control circuit 7 that controls the light source 1 and the photodetector 2.

The light source 1 and the photodetector 2 are arranged side by side. The light source 1 emits light toward a target part 6 of a subject 5. The photodetector 2 detects light emitted from the light source 1 and reflected by the target part 6. The control circuit 7 controls the emission of light by the light source 1 and the detection of light by the photodetector 2. The biological measuring device 17 according to Embodiment 1 includes a signal processing circuit 30 that processes electric signals (hereinafter simply referred to as “signals”) that are outputted from the photodetector 2. The signal processing circuit 30 performs computations based on a plurality of signals outputted from the photodetector 2 and thereby generates information about blood flow in the interior of the target part 6.

The target part 6 according to Embodiment 1 is a forehead part of the subject 5. Information on brain blood flow can be acquired by irradiating the forehead part with light and detecting the resulting scattered light. The “scattered light” includes reflected scattered light and transmitted scattered light. In the following description, the reflected scattered light is sometimes simply referred to as “reflected light”.

Present in the forehead, which is the target part 6, are the scalp (approximately 3 to 6 mm thick), the skull (approximately 5 to 10 mm thick), the cerebrospinal fluid layer (approximately 2 mm thick), and the brain tissue, starting from the surface. The ranges of thicknesses in parentheses mean that there are differences between individuals. Blood vessels are present in the scalp and in the brain tissue. Therefore, blood flow in the scalp is called “scalp blood flow”, and blood flow in the brain tissue is called “brain blood flow”. In a brain function measurement, a measurement object is a target part where there are blood flow distributions both near the surface of and in the interior of the scalp.

A living body is a scatterer. A portion of light 8 emitted toward the target part 6 returns as directly-reflected light 10a to the biological measuring device 17. Another portion of the light enters the interior of the target part 6 and are diffused, and a portion of it is absorbed. The light having entered the interior of the target part 6 turns into internally-scattered light 9a including information on blood flow near the surface that is present approximately 3 to 6 mm deep in the scalp from the surface, i.e. scalp blood flow, internally-scattered light 9b including information on blood flow that is present in a range of depth of approximately 10 to 18 mm from the surface, i.e. brain blood flow, or the like. The internally-reflected light 9a and the internally-reflected light 9b return to the biological measuring device 17 as reflected scattered light 10b from near the surface and as reflected scattered light 11 from the interior, respectively. The directly-reflected light 10a, the reflected scattered light 10b from near the surface, and the reflected scattered light 11 from the interior are detected by the photodetector 2.

It takes the shortest time, the second shortest time, and the longest time for the directly-reflected light 10a, the reflected scattered light 10b from near the surface, and the reflected scattered light 11 from the interior, respectively, to arrive at the photodetector 2 after being emitted from the light source 1. Among them, the component required to be detected at a high S/N ratio is the reflected scattered light 11 from the interior, which has the information on brain blood flow.

It should be noted that the transmitted scattered light, as well as the reflected scattered light, may be used in carrying out a biological measurement other than a brain blood flow measurement. In a case where information on blood other than brain blood flow is acquired, the target part 6 may be a part other than the forehead (e.g. an arm, a leg, or the like). In the following description, unless otherwise noted, the target part 6 is the forehead. The subject 5 is a human, but may alternatively be a non-human animal having skin and having a hairless part. The term “subject” as used herein means specimens in general including such animals.

The light source 1 emits light of, for example, not shorter than 650 nm to not longer than 950 nm. This wavelength range is included in a wavelength range of red to near infrared radiation. The aforementioned wavelength range is called “biological window” and known to be low in absorptance in the body. The light source 1 according to Embodiment 1 is described as one that emits light falling within the aforementioned wavelength range, but light falling within another wavelength range may be used. The term “light” as used herein means not only visible light but also infrared radiation.

In a visible light region of shorter than 650 nm, absorption by hemoglobin (HbO₂ and Hb) is high, and in a wavelength range of longer than 950 nm, absorbance by water is high. Meanwhile, in a wavelength range of not shorter than 650 nm or longer to not longer than 950 nm, the absorption coefficients of hemoglobin and water are comparatively low and the scattering coefficients of hemoglobin and water are comparatively high. Therefore, light falling within the wavelength range of not shorter than 650 nm to not longer than 950 nm is subjected to strong scattering after entering the body and returns to the body surface. This makes it possible to efficiently acquire information on the interior of the body. Accordingly, Embodiment 1 mainly uses light falling within the wavelength range of not shorter than 650 nm to not longer than 950 nm.

The light source 1 may be a laser light source, such as a laser diode (LD), that repeatedly emits light pulses. In a case where the subject 5 is a human as in the case of Embodiment 1, the impact of the light 8 on the retina is considered. In a case where a laser light source is used as the light source 1, a laser light source that satisfies Class 1 of laser safety standards devised by each country is selected, for example. In a case where Class 1 is satisfied, light of such low illuminance that the accessible emission limit AEL falls below 1 mW is emitted toward the part being test 6 of the subject 5. Since the light is of low illuminance, the sensitivity of the photodetector 2 is not enough in many cases. In that case, light pulses are repeatedly emitted. It should be noted that the light source 1 per se does not need to satisfy Class 1. For example, light is diffused or attenuated by placing an element such as a diffusing plate or an ND filter between the light source 1 and the target part 6. In this way, Class 1 of the laser safety standards may be satisfied.

An optical element such as a lens may be provided on an emission surface of the light source 1 to adjust the degree of divergence of the light 8. Furthermore, an optical element such as a lens may be provided on a light-receiving surface side of the photodetector 2 to adjust the rate of extraction of reflected scattered light that is received.

The light source 1 is not limited to a laser light source but may be another type of light source such as a light-emitting diode (LED). Widely useable examples of the light source 1 include a semiconductor laser, a solid laser, a fiber laser, a super luminescent diode, an LED, and the like.

The light source 1 can start and stop the emission of a light pulse and change light powers in accordance with instructions from the control circuit 7. This allows almost any light pulse to be generated from the light source 1.

In order to quantify the light amounts of the directly-reflected light 10a, the reflected scattered light 10b, and the reflected scattered light 11, the inventors ran a simulation of a light pulse response assuming, as the target part 6, a phantom mimicking the head of a typical Japanese. Specifically, the inventors calculated through a Monte Carlo analysis a time distribution of a light power, i.e. a light pulse response, that is detected by the photodetector 2 in a case where a light pulse is emitted toward the target part 6 located at a distance of, for example, 15 cm from the light source 1.

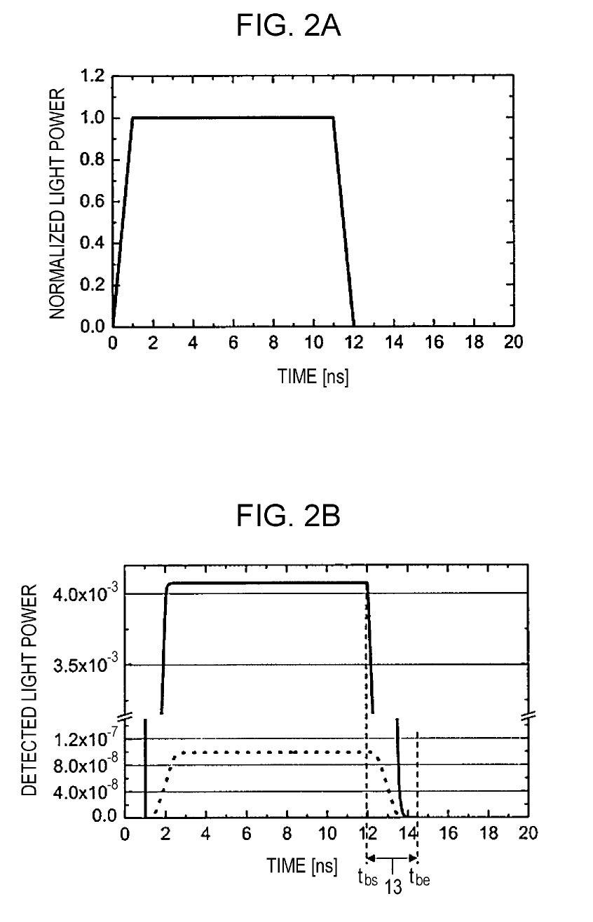

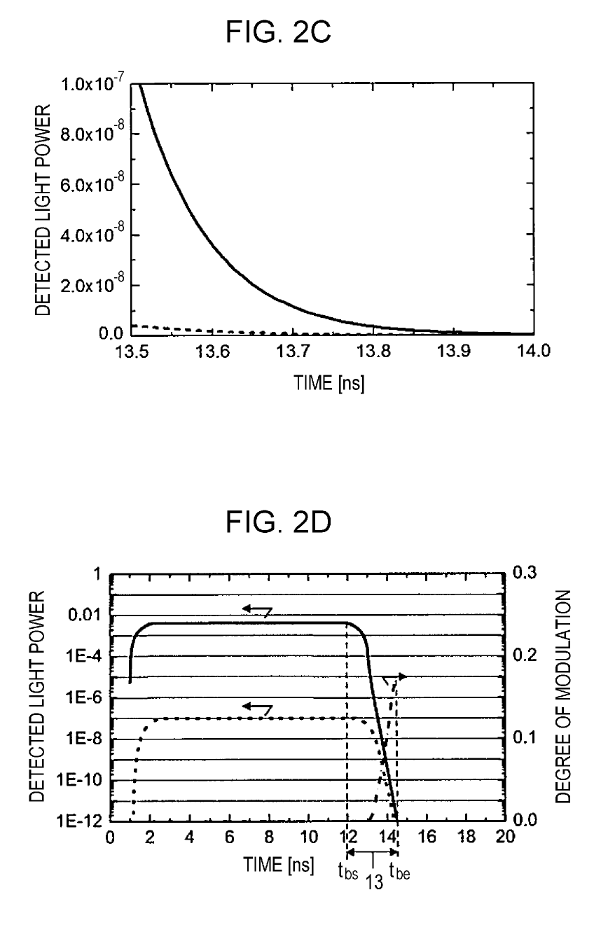

FIG. 2A is a diagram showing an example of a time distribution of a single light pulse that is emitted light. In this example, the light pulse has a wavelength λ of 850 nm and a full width at half maximum of 11 ns. This single light pulse has a typical trapezoidal shape whose rising and falling times are each 1 ns. Assume that the emission of the single light pulse starts at a point in time t=0 and completely stops at t=12 ns.

Since the velocity of light c is 300000 km/s and the distance from the light source 1 to the target part 6 is 15 cm, the point in time at which the light 8 arrives at the surface of the target part 6 is expressed as t=0.5 ns. The point in time at which the light 8 arrives at a surface of the photodetector 2 after being directly reflected by the surface of the target part 6 and turning into the directly-reflected light 10a is expressed as t=1 ns. Therefore, the point in time T_{d} at which the light is detected on the photodetector 2 is expressed as T_{d}≤1 ns.

The biological measuring device 17 measures the amount of change in light amount of the reflected scattered light 11 from the interior of the target part 6 on the basis of changes in oxyhemoglobin concentration and deoxyhemoglobin concentration in the brain blood flow. The brain tissue has an absorber whose absorption coefficient and scattering coefficient vary according to changes in brain blood flow. In a stationary state, it is possible to model the interior of the brain as uniform brain tissue and conduct a Monte Carlo analysis. The term “changes in blood flow” as used herein means temporal changes in blood flow.

FIG. 2B is a diagram showing time distributions of a total light power (solid line) in a stationary state and a power (dashed line) of light having passed through a region of change in brain blood flow. FIG. 2C is a diagram showing time distributions of the total light power (solid line) in the stationary state and the power (dashed line) of the light having passed through the region of change in brain blood flow. FIG. 2C is a diagram showing time distributions, during a falling period, of the total light power (solid line) in the stationary state and the power (dashed line) of the light having passed through the region of change in brain blood flow. The falling period means a period from the start of a decrease in light power to the end of the decrease. FIG. 2D is a diagram showing time distributions of the total light power (solid line) in the stationary state, the power (dashed line) of the light having passed through the region of change in brain blood flow, and a degree of modulation (chain line). The degree of modulation means a value obtained by dividing, by the total amount of light in the stationary state, the amount of light having passed through the region of change in brain blood flow. In each of FIGS. 2B and 2C, the vertical axis is expressed by a linear display, and in FIG. 2D, the vertical axis is expressed by a logarithmic display.

The amount of light having passed through the region of change in brain blood flow, which is included in the total amount of light that is detected by the photodetector 2, is only approximately 2×10⁻⁵. That is, in a case where the light 8, which is a light pulse, is emitted, the total amount of light is detected by the photodetector 2, and a change therein is detected, a component included in the light amount thus detected that indicates changes in brain blood flow is so small as to be negligible. On the other hand, the directly-reflected light 10a is constant in light amount and has a reflectance of, for example, approximately 4%. This makes it possible to detect changes in light amount of the directly-reflected light 10a from near the surface, i.e. changes in scalp blood flow.

Let it be assumed that t_{bs} is the point in time at which the light power starts to decrease on the photodetector 2 and t_{be} is the point in time at which the light power completely decreases to a noise level. As shown in FIG. 2D, it is found that the proportion of signals that indicate changes in brain blood flow becomes higher in a falling period 13 of light t_{bs}≤t≤t_{be}. As the second half of the falling period 13 of light passes, the light amount decreases and noise increases accordingly. However, the degree of modulation becomes higher. Of the light falling period 13 of t_{bs}≤t≤t_{be}, the amount of light at and after t=13.5 ns, for example, is approximately 1/100 of the total detected light amount of the light pulse. In a case where light arriving during the falling period 13 is detected with use of the function of an electronic shutter of the photodetector 2, the proportion of the light having passed through the region of change in brain blood flow increases to 7% of the total amount of light detected at and after t=13.5 ns. This makes it possible to sufficiently acquire signals that indicate changes in brain blood flow. Without use of the electronic shutter, the proportion of changes in brain blood flow is approximately 2×10⁻⁵.

Therefore, signals that indicate changes in brain blood flow can be detected by receiving, through the photodetector 2, a component of the light 11 included in the falling period 13 of light from the target part 6 and detecting changes in light amount thereof.

With reference to the aforementioned principle of measurement of changes in scalp blood flow and brain blood flow, the emission of a light pulse and the detection of light in the biological measuring device 17 according to Embodiment 1 is described.

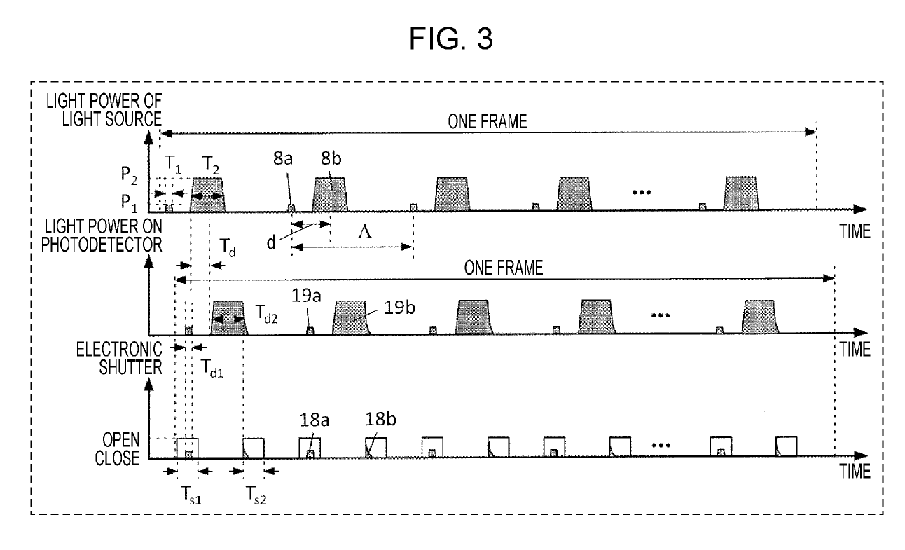

FIG. 3 is a diagram schematically showing a time distribution (upper row) of first and second light pulses, a time distribution (middle row) of a light power on the photodetector 2 in a case where the first and second light pulses are emitted, and the timing and charge storage (lower row) of the electronic shutter according to Embodiment 1 of the present disclosure.

In the biological measuring device 17 according to Embodiment 1, the control circuit 7 causes the light source 1 to emit at least one first light pulse and at least one second light pulse at different timings, respectively. The control circuit 7 causes the photodetector 2 to detect a first component and output a first electric signal representing the first component thus detected. The first component is a component of light included in at least one first reflected light pulse returning from the target part 6. The control circuit 7 causes the photodetector 2 to detect a second component and output a second electric signal representing the second component thus detected. The second component is a component of light included in a falling period of at least one second reflected light pulse returning from the target part 6.

As shown in the upper row of FIG. 3, the light source 1 emits a first light pulse 8a and a second light pulse 8b in sequence. The first light pulse 8a has a pulse width T₁ and a maximum light power value P₁, and the second light pulse 8b has a pulse width T₂ and a maximum light power value P₂. The term “pulse width” as used herein means the full width at half maximum of a pulse waveform. A time interval from the center of the first light pulse 8a to the center of the second light pulse 8b is d.

As shown in the middle row of FIG. 3, light 19a corresponding to the first light pulse 8a and returning from the target part 6 has a pulse width T_{d1}, which is substantially the same as T₁. Similarly, light 19b corresponding to the second light pulse 8b and returning from the target part 6 has a pulse width T_{d2}, which is substantially the same as T₂. As shown in the middle row of FIG. 3, the light 19a and light 19b returning from the target part 6 have shapes that become wider toward the skirts due to the occurrence of time delays under the influence of internal scattering.

The photodetector 2 includes a photoelectric conversion section 3 and a charge storage section (hereinafter referred to as “storage section”). The photoelectric conversion section 3 photoelectrically converts a component of the light 19a corresponding to the first light pulse 8a and returning from the target part 6 and a component of light, included in the falling period 13, of the light 19b corresponding to the second light pulse 8b and returning from the target part 6. The storage section accumulates a first signal charge 18a and a second signal charge 18b.

In Embodiment 1, the pulse width T₁ of the first light pulse 8a is shorter than the pulse width T₂ of the second light pulse 8b (T₁<T₂). For example, T₁=1 to 3 ns and T₂=11 to 22 ns. The maximum light power value P₁ of the first light pulse 8a is lower than the maximum light power value P₂ of the second light pulse 8b (P₁<P₂). For example, P₁/P₂=0.01 to 0.1. Alternatively, the first light pulse 8a and the second light pulse 8b may be the same in maximum light power, and the pulse width of the first light pulse 8a may be smaller than the pulse width of the second light pulse 8b.

In a case where the target part 6 is the forehead of a human, each light pulse may enter the eyes. For this reason, the first light pulse 8a and the second light pulse 8b may be emitted, for example, with such a low power as to satisfy Class 1. In order to satisfy the sensitivity of the photodetector 2, the first light pulse 8a and the second light pulse 8b may be repeatedly emitted. For example, a pair of the first light pulse 8a and the second light pulse 8b may be repeatedly emitted 10000 times to 1000000 times in a time cycle Λ of approximately 55 ns to 110 ns. This allows one frame to be composed. A moving image can be composed by arranging frames. It should be noted that the first light pulse 8a and the second light pulse 8b need only be included in an identical frame period, the order of the first light pulse 8a and the second light pulse 8b may be changed.

Further, the time interval d from the center of a first light pulse 8a to the center of a second light pulse 8b that is emitted immediately after the first light pulse 8a, may be made shorter than a time interval (Λ−d) from the center of a second light pulse 8b to the center of a first light pulse 8a that is emitted immediately after the second light pulse 8b. Setting the time interval d as appropriate makes it possible to use the after-mentioned electronic shutter to substantially equalize lengths of time that two storage sections 4a and 4b accumulate electric charge. This brings about an effect of making control easy.

It is possible, without imposing a Class 1 limitation, to measure biological information other than brain blood flow with a high light power or measure biological information with use of a highly sensitive photodetector such as an avalanche photodiode. In that case, the emission of each of the first light pulse 8a and the second light pulse 8b does not necessarily need to be repeated more than once. For example, biological information may be detected by irradiating the target part 6 once with each of the first light pulse 8a and the second light pulse 8b.

In the biological measuring device 17 according to Embodiment 1, the photodetector 2 includes the electronic shutter, which switches between storing signal charge and not storing signal charge, and the plurality of storage sections 4a and 4b. The electronic shutter is a circuit that controls the storage and discharge of signal charge generated by the photoelectric conversion section 3.

The photoelectric conversion section 3 photoelectrically converts the light 19a returning from the target part 6 as a result of emitting the first light pulse 8a. After that, the storage section 4a is selected in accordance with control signals 16a, 16b, and 16e from the control circuit 7; that is, the electronic shutter is opened and the first signal charge 18a is accumulated for a period of time T_{S1} of, for example, 11 to 22 ns. After the period of time T_{S1} has elapsed, a drain 12 is selected in accordance with the control signals 16a, 16b, and 16e from the control circuit 7; that is, the electronic shutter is closed and electric charge from the photoelectric conversion section 3 is released.

Similarly, the photoelectric conversion section 3 photoelectrically converts the component of the reflected scattered light 11, which is light included in the falling period 13 of the light 19b corresponding to the second light pulse 8b and returning from the target part 6. After that, the other storage section 4b is selected in accordance with the control signals 16a, 16b, and 16e, and the second signal charge 18b is accumulated for a period of time T_{S2} of, for example, 11 to 22 ns. After the period of time T_{S2} has elapsed, the drain 12 is selected in accordance with the control signals 16a, 16b, and 16e from the control circuit 7, and electric charge from the photoelectric conversion section 3 is released.

Therefore, the component of the light 19a corresponding to a repetitive pulse train of the first light pulses 8a and returning from the target part 6 is accumulated as one frame of the first signal charge 18a in the storage section 4a by the photoelectric conversion. After the end of one frame, the first signal charge 18a is outputted as a first electric signal 15a to the control circuit 7. The first electric signal 15a includes the information on scalp blood flow.

The component of the reflected scattered light 11, which is light included in the falling period 13 of the light 19b corresponding to a repetitive pulse train of the second light pulses 8b and returning from the target part 6, is accumulated as one frame of the second signal charge 18b in the storage section 4b by the photoelectric conversion. After the end of one frame, the second signal charge 18b is outputted as a second electric signal 15b to the control circuit 7. The second electric signal 15b includes the information on scalp blood flow as well as the information on brain blood flow.

After the emission of the first light pulse 8a and the second light pulse 8b, ambient noise may be measured by keeping the electronic shutter open and closed for the same length of time and the same number of times in the absence of light emission. The S/N ratios of the signals can be improved by subtracting the value of the ambient noise from each of the signal values. T_{S1} and T_{S2} may be the same as or different from each other. If T_{S1}=T_{S2}, it is only necessary to measure the ambient noise once by keeping the electronic shutter open for T_{S1}. This makes it possible to omit to carry out a second measurement of the ambient noise by keeping the electronic shutter open for T_{S2}.

In Embodiment 1, the pulse width T₁ of the first light pulse 8a is shorter than the period of time T_{S1} during which to accumulate the first signal charge 18a (T₁<T_{S1}). In this case, there may be fluctuation (jitter) in the timing of emission of the first light pulse 8a or the length of time that the electronic shutter is kept open or closed. Furthermore, there may be a slight variation in distance from the target part 6 to the biological measuring device 17. Making T₁ shorter than T_{S1} makes it possible to substantially cancel or reduce the variation, thus making it possible to hold the accumulated charge amount constant. That is, this brings about an effect of making it possible to improve the jitter margins and reduce the influence of a motion of the target part 6 on the detection of blood flow near the surface.

The configuration of the photodetector 2 as shown in FIG. 1B is equivalent to one pixel. This makes it possible to acquire information about averaged blood flow in the target part 6.

Alternatively, it is possible to use, as the photodetector 2, an image sensor including, for each pixel, a photoelectric conversion section 3, a storage section, and an electronic shutter that switches between storing signal charge and not storing signal charge in the storage section. In this case, the photodetector 2 is an image sensor having a plurality of photodetection cells arrayed two-dimensionally. Each of the photodetection cells accumulates, as the first signal charge 18a, a component of light included in a first light pulse and accumulates, as the second signal charge 18b, a component of light included in a falling period of a second light pulse. Furthermore, each of the photodetection cells outputs, as the first electric signal 15a, an electric signal representing a total amount of the first signal charge 18a thus accumulated and outputs, as the second electric signal 15b, an electric signal representing a total amount of the second signal charge 18b thus accumulated. This makes it possible to acquire biological information about the blood flow of the target part 6 as a moving image including a plurality of frames.

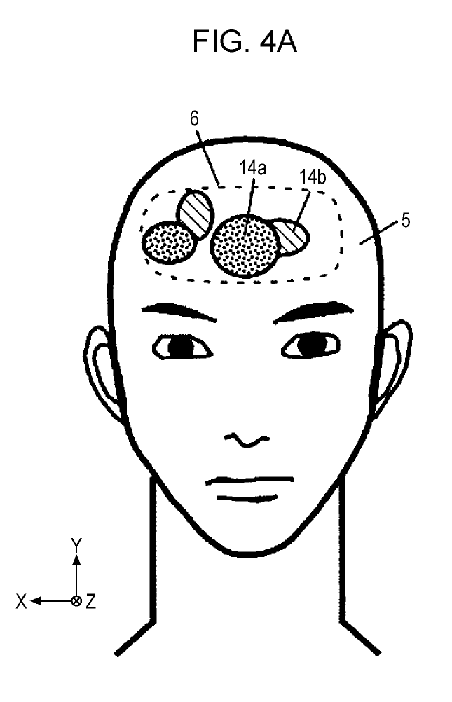

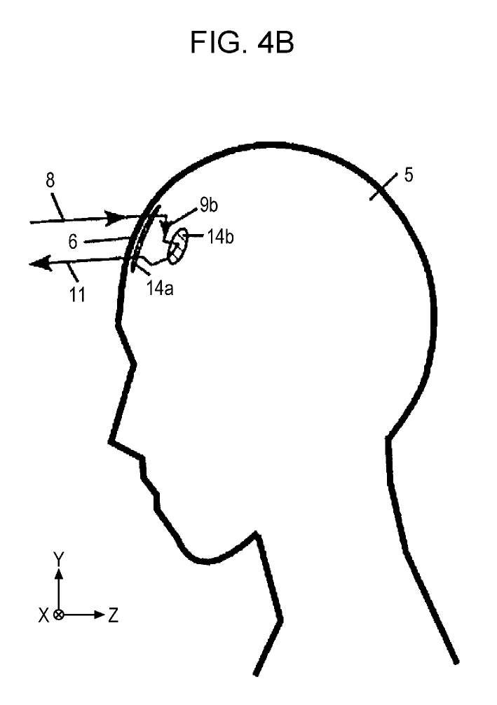

Next, the superimposition of the information on brain blood flow and the information on scalp blood flow onto the second electric signal 15b is described with reference to FIGS. 4A and 4B.

FIG. 4A is a front view showing changes in blood flow that are present in the surface and interior of the target part 6. FIG. 4B is a Y-Z plane cross-sectional view showing the changes in blood flow that are present in the surface and interior of the target part 6. FIGS. 4A and 4B show a scalp blood flow region 14a and a brain blood flow region 14b. The scalp blood flow region 14a, located at a depth of, for example, approximately 3 to 6 mm in the epidermis from the surface of the target part 6, which is a forehead, is a region of change in blood flow near the surface (i.e. scalp blood flow). The brain blood flow region 14b is a region of change in blood flow in the interior (i.e. brain blood flow) at a depth of approximately 10 to 18 mm from the surface. Attention is paid to the optical path through which the light 8 enters the target part 6 and is detected as the internally-scattered light 9b by the photodetector 2. The internally-scattered 9b, albeit depending on a blood flow distribution, passes through the scalp blood flow region 14a first and then is scattered or absorbed to pass through the brain blood flow region 14b. Furthermore, the internally-scattered 9b is repeatedly scattered or absorbed to pass through the scalp blood flow region 14a again and come out of the target part 6. That is, the information on scalp blood flow is superimposed on the information on brain blood flow included in the falling period 13 of the light 19b corresponding to the repetitive pulse train of the second light pulses 8b and returning from the target part 6. This causes deterioration in S/N ratio of the information on brain blood flow. The information on brain blood flow is influenced by the scalp blood flow region 14a being superimposed thereonto on an outward path. However, the influence is made smaller by scattering or absorption on the outward and return optical paths in the living body. Therefore, the information on brain blood flow is greatly influenced by the scalp blood flow region 14a being superimposed thereonto on a return path.

Next, a method for acquiring a distribution that indicates changes in blood flow in the target part 6 is described.

First, the control circuit 7 causes the photodetector 2, which is an image sensor, to output the following first to fourth image signals. The first image signal represents a two-dimensional distribution of the total amount of the first signal charge 18a accumulated in the plurality of photodetection cells during a first period. The second image signal represents a two-dimensional distribution of the total amount of the second signal charge 18b accumulated in the plurality of photodetection cells during a second period that is identical to or different from the first period. The third image signal represents the two-dimensional distribution of the total amount of the first signal charge 18a accumulated in the plurality of photodetection cells during a third period preceding the first period. The fourth image signal represents the two-dimensional distribution of the total amount of the second signal charge 18b accumulated in the plurality of photodetection cells during a fourth period preceding the second period.

Next, the signal processing circuit 30 receives the first to fourth image signals from the photodetector 2, which is an image sensor. After that, the signal processing circuit 30 generates a first difference image representing a difference between an image represented by the first image signal and an image represented by the third image signal and generates a second difference image representing a difference between an image represented by the second image signal and an image represented by the fourth image signal.

The first difference image is equivalent to a distribution that indicates changes in scalp blood flow in the target part 6, and the second difference image is equivalent to a distribution that indicates changes in scalp blood flow and brain blood flow in the target part 6. It is assumed herein that the first and second difference images are images that represent the absolute values of the differences. When the signal processing circuit 30 receives the third and fourth image signals only once and repeatedly receives the first and second image signals every one-frame cycle, a moving image of a distribution that indicates changes in blood flow in the target part 6 is obtained.

As shown in the example of FIG. 3, the first and second periods may be the same as each other, and the third and fourth periods may be the same as each other. As will be mentioned later, the second period may be a frame period that follows the first period, and the fourth period may be a frame period that follows the third period.

Next, a method for improving the S/N ratio of the information on brain blood flow is described.

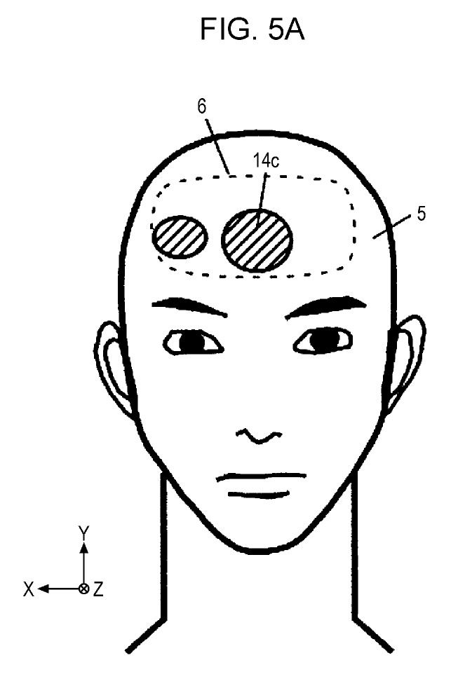

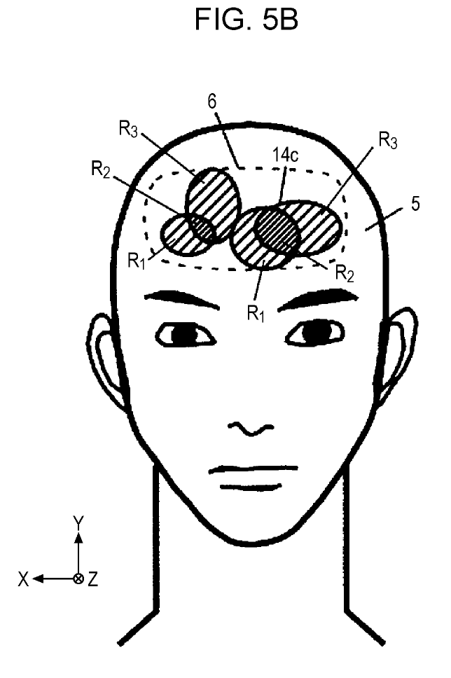

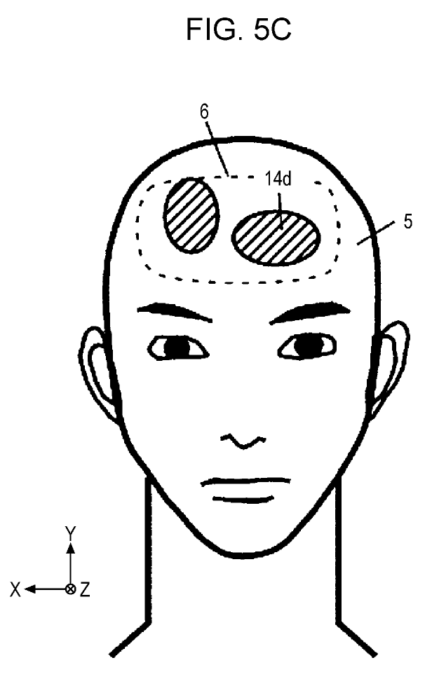

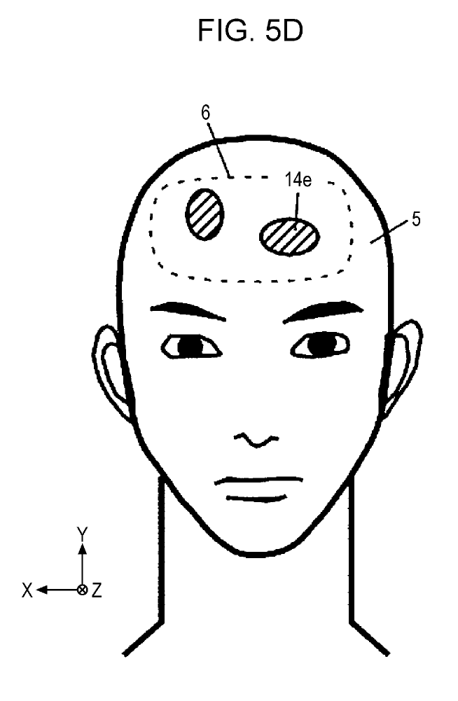

FIG. 5A is a diagram schematically showing changes in blood flow in the surface of the target part 6 as detected by the first light pulse. FIG. 5B is a diagram schematically showing changes in blood flow in the surface and interior of the target part 6 as detected by the second light pulse. FIG. 5C is a diagram schematically showing changes in blood flow in the interior of the target part 6 as derived by image computations. FIG. 5D is a diagram schematically showing changes in blood flow in the interior of the target part 6 as derived by further image computations.

As shown in FIG. 5A, the signal processing circuit 30 generates the first difference image, which is equivalent to a distribution 14c that indicates changes in scalp blood flow, in accordance with the first electric signal 15a generated by irradiation with a pulse train of the first light pulses 8a. Next, in accordance with the second electric signal 15b from charge accumulated with a time delay with use of the electronic shutter by irradiation with a pulse train of the second light pulses 8b, the signal processing circuit 30 generates the second difference image, which is equivalent to such a distribution 14c as that shown in FIG. 5B on which the information on scalp blood flow and brain blood flow is superimposed and which indicates changes in blood flow. The distribution 14c in FIG. 5B has a region R₁ that includes the information on scalp blood flow and does not include the information on brain blood flow, a region R₂ that includes the information on both scalp blood flow and brain blood flow, and a region R₃ that includes the information on brain blood flow and does not include the information on scalp blood flow.

The signal processing circuit 30 generates blood flow information on the interior of the target part 6 through computations based on the first electric signal 15, which represents the amount of the first signal charge 18a, and the second electric signal 15b, which represents the amount of the second signal charge 18b. The first signal charge 18a includes blood flow information on the surface of the target part 6, and the second signal charge 18b includes blood flow information on the surface and interior of the target part 6.

Through image computations including subtractions and divisions based on the two two-dimensional images in FIGS. 5A and 5B, the signal processing circuit 30 generates a two-dimensional image of a distribution 14d in FIG. 5C that indicates changes in brain blood flow. For example, a correction is made so that the two distributions become equal in intensity in the region R₁ in FIG. 5B and a region in FIG. 5A that is equivalent to the region R₁. After that, it is only necessary to subtract, from the distribution in FIG. 5B that indicates the blood flow information on the surface and the interior, the distribution in FIG. 5A that indicates the blood flow information on the surface. This gives a distribution that indicates the blood flow information on the interior.

The two-dimensional image in FIG. 5C represents the distribution 14d, which indicates changes in brain blood flow. The distribution 14d, which indicates changes in brain blood flow, is in a spread state due to scattering of brain blood flow in the interior. To address this problem, the signal processing circuit 30 makes an image correction by guessing the scattering state through a diffusion equation or a Monte Carlo analysis. By so doing, the signal processing circuit 30 generates a two-dimensional image of a distribution 14e in FIG. 5D that indicates changes in brain blood flow. This two-dimensional image is a desired distribution that indicates changes in brain blood flow.

In this method, for computations at high S/N ratios, it is possible, for example, to equalize the luminances of regions in the two images in FIGS. 5A and 5B that indicate changes in blood flow in the surface of the target part 6.

A first region in the image of FIG. 5A is a region 14c that is formed by a plurality of pixels having pixel values exceeding a first threshold. Similarly, a second region in the image of FIG. 5B is a region 14c that is formed by a plurality of pixels having pixel values exceeding a second threshold. The first threshold may be equal to or different from the second threshold. M₁ is the average pixel value of a part of the first region in the image of FIG. 5A that overlaps the second region. Similarly, M₂ is the average pixel value of a part of the second region in the image of FIG. 5B that overlaps the first region. In this case, it may be, for example, that M₁=M₂. Since an adjustment is possible with an image correction, M₁ and M₂ may satisfy, for example, 0.1 M₁/M₂≤10. The condition can be attained by adjusting at least one of the pulse widths T₁ and T₂, the maximum power values P₁ and P₂, and the timing of opening of the electronic shutter in the second storage section 4b.

The part of the first region in the example of FIG. 5A that overlaps the second region in the example of FIG. 5B includes the information on scalp blood flow and does not include the information on brain blood flow. Meanwhile, the part of the second region in the example of FIG. 5B that overlaps the first region in the example of FIG. 5A includes part of the information on brain blood flow as well as the information on scalp blood flow. In this case, too, as mentioned above, the ratio of M₂ to M₁ has a margin of approximately one digit. Therefore, there is no problem even if M₂ includes part of the information on brain blood flow.

Alternatively, the light source 1 may include two light-emitting elements. For example, a first one of the light-emitting elements may emit a first light pulse 8a and then a second one of the light-emitting elements may emit a second light pulse 8b. In this configuration, the maximum power value of each of the light-emitting elements may be constant. This makes it easy to control output from the light source 1.

Next, biological measuring devices according to modifications of Embodiment 1 of the present disclosure are described.

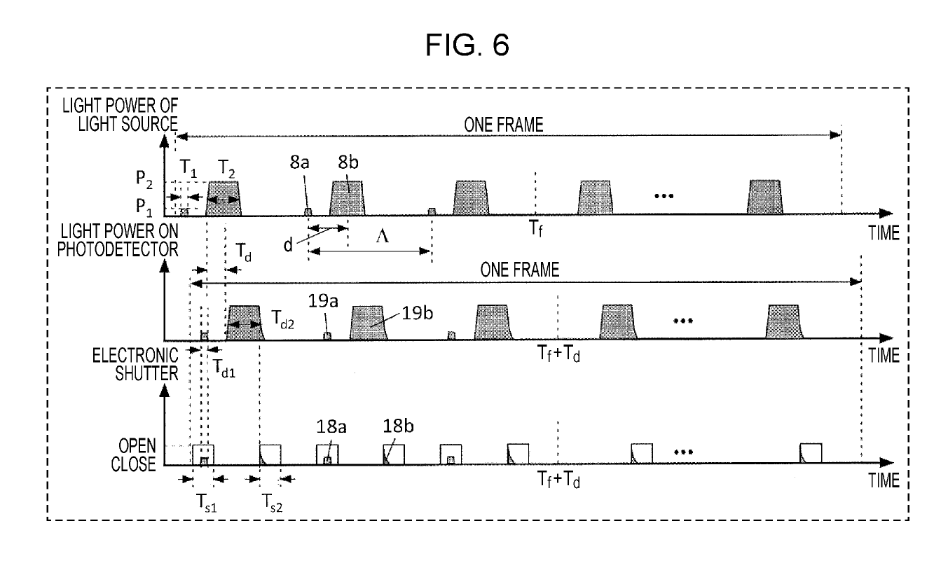

FIG. 6 is a diagram schematically showing a time distribution (upper row) of first and second light pulses, a time distribution (middle row) of a light power on the photodetector 2 in a case where the first and second light pulses are emitted, and the timing and charge storage (lower row) of the electronic shutter according to Modification 1 of Embodiment 1 of the present disclosure.

In this example, as described above, the light source 1 alternately emits the first light pulse 8a and the second light pulse 8b as the light 8 in the first half t<T_{f} of a one-frame period. The light source 1 repeatedly emits only the second light pulse 8b in the second half t>T_{f} of the one-frame period. When, in the first half of the one-frame period, the average pixel value M₁, which is obtained by the first light pulse 8a, exceeds the average pixel value M₂, which is obtained by the second light pulse 8b (M₁>M₂), the average pixel value M₂, which is obtained by the second light pulse 8b, may be increased in the second half of the one-frame period, whereby an adjustment may be made so that M₁ and M₂ become equal average pixel values. Modification 1 is effective when the span of adjustable range of at least one of the intensity and pulse width of each light pulse is small and the amount of electric charge accumulated by the first light pulse 8a per pulse is larger than the amount of electric charge accumulated by the second light pulse 8b per pulse.

Further, the first and second halves of an array of the first light pulses 8a and the second light pulses 8b may be swapped. That is, the light source 1 may repeatedly emit the second light pulse 8b in the first half of a one-frame period and alternately repeatedly emit the first light pulse 8a and the second light pulse 8b in the second half of the one-frame period.

Furthermore, there may be a case where when the span of adjustable range of at least one of the intensity and pulse width of each light pulse is large and the amount of electric charge accumulated by the first light pulse 8a per pulse is smaller than the amount of electric charge accumulated by the second light pulse 8b per pulse. In that case, the light source 1 may alternately repeatedly emit the first light pulse 8a and the second light pulse 8b in the first half of a one-frame period and repeatedly emit the first light pulse 8a in the second half of the one-frame period.

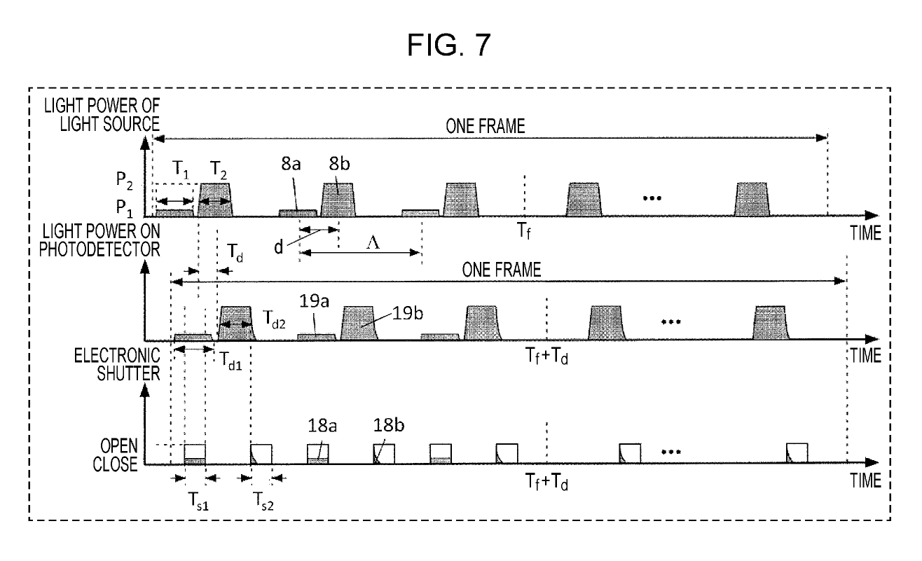

FIG. 7 is a diagram schematically showing a time distribution (upper row) of first and second light pulses, a time distribution (middle row) of a light power on the photodetector 2 in a case where the first and second light pulses are emitted, and the timing and charge storage (lower row) of the electronic shutter according to Modification 2 of Embodiment 1 of the present disclosure.

Modification 2 differs from Modification 1 of Embodiment 1 in that the pulse width T₁ of the first light pulse 8a is longer than the period of time T_{S1} during which the electronic shutter is kept open (T₁>T_{S1}). In this case, there may be fluctuation (jitter) in the timing of emission of the first light pulse 8a or the length of time that the electronic shutter is kept open and closed. Furthermore, there may be a slight variation in distance between the target part 6 and the biological measuring device 17. Making T₁ longer than T_{S1} makes it possible to substantially cancel or reduce the variation, thus making it possible to hold the accumulated charge amount constant. That is, this brings about an effect of making it possible to improve the jitter margins and reduce the influence of a motion of the target part 6 on the detection of blood flow near the surface.

In this case, electric charge may be accumulated for the same length of time as the total period of time T_{S1} during which the electronic shutter is kept open. This makes it possible to make the average pixel value M₁, which is obtained by the first light pulse 8a, greater than it is in Modification 1 of Embodiment 1. Therefore, it is effective to increase the average pixel value M₂, which is obtained by the second light pulse 8b by repeatedly emitting only the second light pulse 8b in the second half of a one-frame period.

In this example, the first and second halves of an array of the first light pulses 8a and the second light pulses 8b may be swapped. That is, the light source 1 may repeatedly emit the second light pulse 8b in the first half of a one-frame period and alternately repeatedly emit the first light pulse 8a and the second light pulse 8b in the second half of the one-frame period.

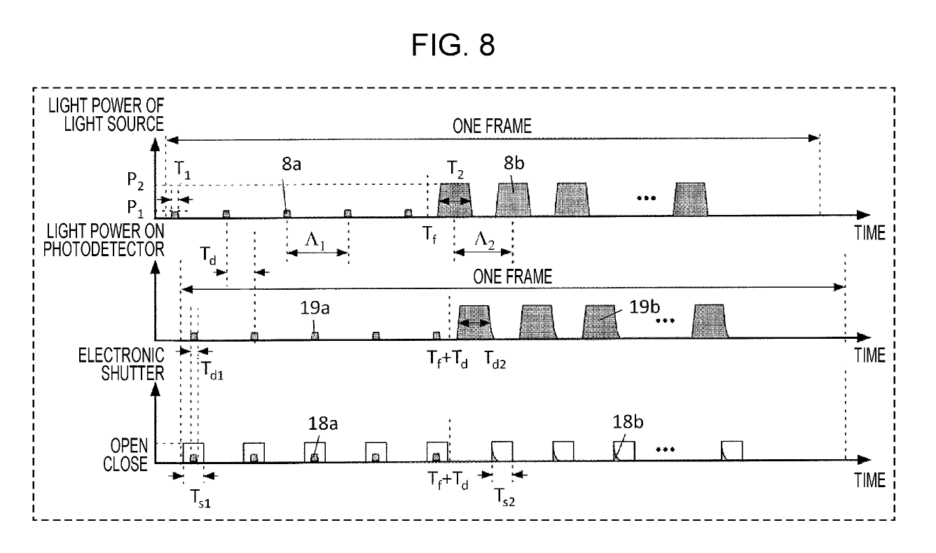

FIG. 8 is a diagram schematically showing a time distribution (upper row) of first and second light pulses, a time distribution (middle row) of a light power on the photodetector 2 in a case where the first and second light pulses are emitted, and the timing and charge storage (lower row) of the electronic shutter according to Modification 3 of Embodiment 1 of the present disclosure.

In this example, the light source 1 repeatedly emits the first light pulse 8a in the first half t<T_{f} of a one-frame period and repeatedly emits the second light pulse 8b in the second half t>T_{f}. By controlling the number of repetitions of each of the first light pulse 8a and the second light pulse 8b, an adjustment may be made so that the average pixel values M₁ and M₂ of the one-frame period, which are obtained by the first light pulse 8a and the second light pulse 8b, satisfy M₁=M₂ or 0.1≤M₁/M₂≤10. This makes it only necessary to switch between the storage sections 4a and 4b only once in each of the first and second halves of a one-frame period, thus making control easy.

Further, the first and second halves of an array of the first light pulses 8a and the second light pulses 8b may be swapped. That is, the light source 1 may repeatedly emit the second light pulse 8b in the first half of a one-frame period and repeatedly emit the first light pulse 8a in the second half of the one-frame period.

### Embodiment 2

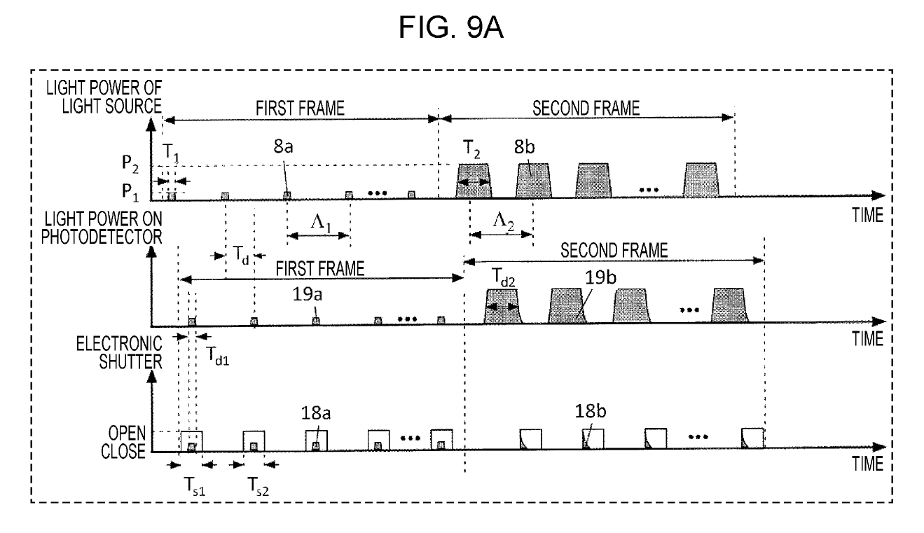

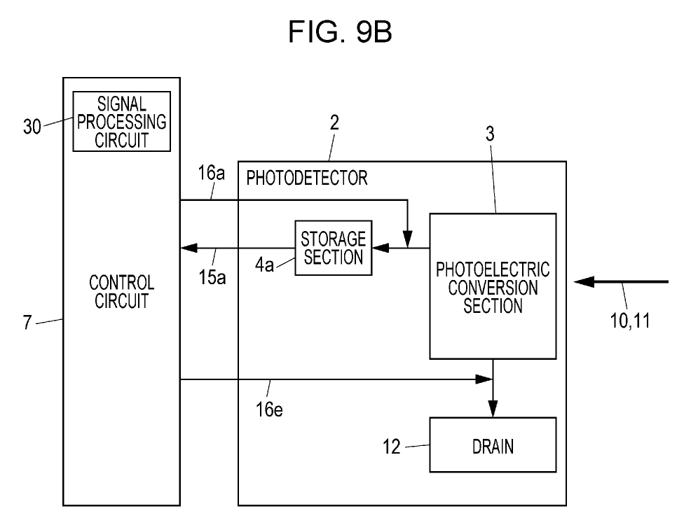

Next, a biological measuring device according to Embodiment 2 of the present disclosure is described with reference to FIGS. 9A and 9B with a focus on differences from the biological measuring device according to Embodiment 1.

FIG. 9A is a diagram schematically showing a time distribution (upper row) of first and second light pulses, a time distribution (middle row) of a light power on the photodetector 2 in a case where the first and second light pulses are emitted, and the timing and charge storage (lower row) of an electronic shutter according to Embodiment 2 of the present disclosure. FIG. 9B is a diagram schematically showing an internal configuration of the photodetector 2 according to Embodiment 2 of the present disclosure and the flow of electric signals and control signals.

The biological measuring device 17 according to Embodiment 2, the control circuit 7 causes the light source 1 to repeatedly emit a first light pulse during a first frame period and causes the photodetector 2 to repeatedly accumulate, in synchronization with the emission of the first light pulse, a first signal charge corresponding to at least part of the first light pulse. The control circuit 7 causes the light source 1 to repeatedly emit a second light pulse during a second frame period that follows the first frame period and causes the photodetector 2 to repeatedly accumulate, in synchronization with the emission of the second light pulse, a second signal charge corresponding to at least part of a falling period of a second reflected light pulse returning from the target part 6.

The biological measuring device according to Embodiment 2 differs from the biological measuring device according to Embodiment 1 in that the photodetector 2 includes only one storage section 4a and the first light pulse 8a and the second light pulse 8b are included in different frame periods, respectively. The light source 1 repeatedly emits the first light pulse 8a during the first frame period and repeatedly emits the second light pulse 8a during the second frame period that follows the first frame period.

In the case of execution of the aforementioned method for acquiring a distribution that indicates changes in blood flow in the target part 6 in Embodiment 2, the first period is equivalent to the first frame period, the second period is equivalent to the second frame period, and the fourth period is equivalent to a frame period that follows the third period. As mentioned above, the distribution that indicates changes in scalp blood flow in the target part 6 and the distribution that indicates changes in scalp blood flow and brain blood flow in the target part 6 can be obtained from the first to fourth electric signals. A moving image may be acquired by repeating this operation. The first and second frame periods may be swapped so that the second light pulse 8b is repeatedly emitted during the first frame period and the first light pule 8a is repeatedly emitted during the second frame period.

Since the photodetector 2 includes only one storage section, it is unnecessary to switch between storage sections. This brings about an effect of making the configuration simple and making control easy. It should be noted that in a case where the photodetector 2 includes a plurality of storage sections, it is only necessary to use one of them.

### Embodiment 3

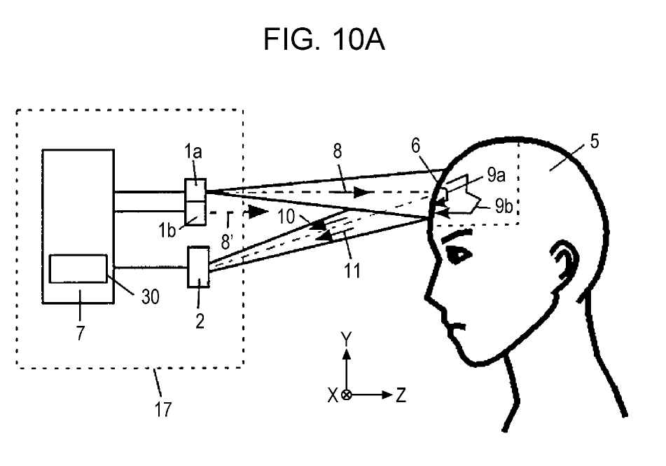

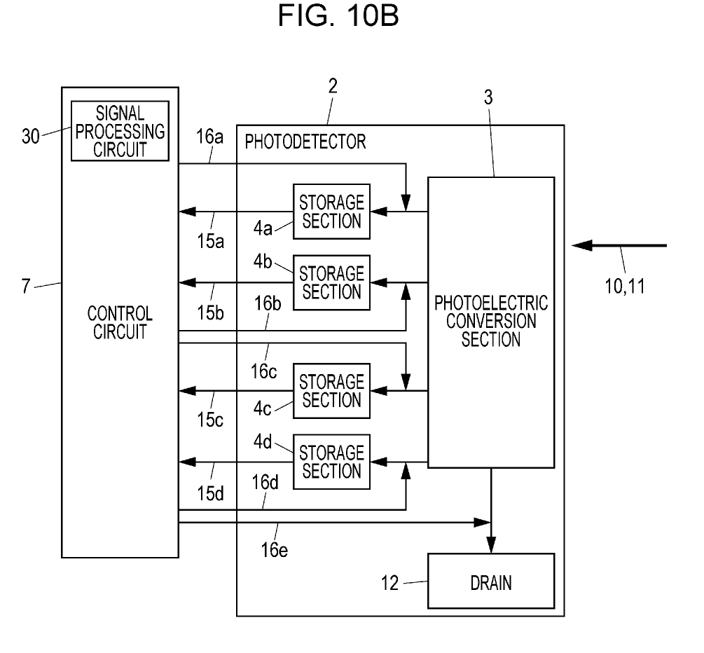

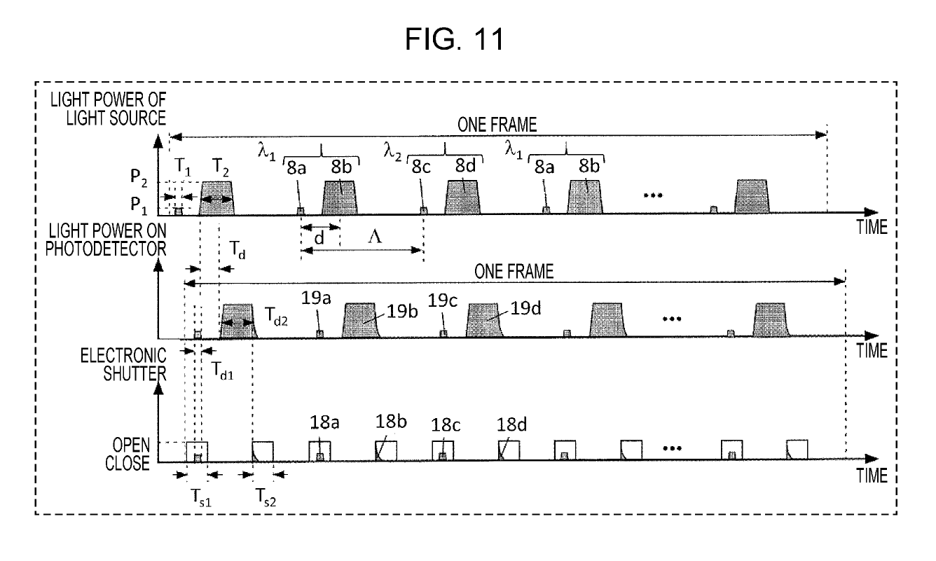

Next, a biological measuring device according to Embodiment 3 of the present disclosure is described with reference to FIGS. 10A, 10B, and 11 with a focus on differences from the biological measuring device according to Embodiment 1.

FIG. 10A is a schematic view for explaining a configuration of a biological measuring device 17 according to Embodiment 3 of the present disclosure and the way in which a biological measurement is carried out. FIG. 10B is a diagram schematically showing an internal configuration of a photodetector 2 according to Embodiment 3 of the present disclosure and the flow of electric signals and control signals.

FIG. 11 is a diagram schematically showing a time distribution (upper row) of first and second light pulses, a time distribution (middle row) of a light power on the photodetector 2 in a case where the first and second light pulses are emitted, and the timing and charge storage (lower row) of an electronic shutter according to Embodiment 3 of the present disclosure.

The biological measuring device 17 according to Embodiment 3 differs from the biological measuring device 17 according to Embodiment 1 in that the light source 1 is a multiwavelength light source that emits at least two wavelengths of light and emits first light pulses 8a and 8c and second light pulses 8b and 8d for each wavelength in sequence.

The light source 1 is composed of a plurality of light-emitting elements 1a and 1b arranged side by side in a Y direction. The light-emitting elements 1a and 1b are for example laser chips. The absorptance of oxyhemoglobin and deoxyhemoglobin varies, for example, at wavelengths of λ₁=750 nm and λ₂=850 nm. Therefore, computing two electric signals respectively obtained by using these two wavelengths makes it possible to measure the proportions of oxyhemoglobin and deoxyhemoglobin in the target part 6.

When the target part 6 is a forehead area of the head of a living body, the amount of change in brain blood flow in the frontal lobe, the amounts of change in oxyhemoglobin concentration and deoxyhemoglobin concentration, or the like can be measured. This makes sensing of information such as emotions possible. For example, in a centered state, there occur an increase in brain blood flow volume, an increase in amount of oxyhemoglobin, and the like.

Various combinations of wavelengths are possible. At a wavelength of 805 nm, the rates of absorption of oxyhemoglobin and deoxyhemoglobin become equal. Therefore, in view of the biological window, for example, a wavelength of not shorter than 650 nm and shorter than 805 nm and a wavelength of longer than 805 nm and not longer than 950 nm may be combined. Furthermore, it is possible to use three wavelengths by using a wavelength of 805 nm in addition to the two wavelengths. In a case where three wavelengths of light are used, three laser chips are needed; however, since information on the third wavelength is obtained, utilizing the information may make computations easy.

The photodetector 2 of the biological measuring device 17 according to Embodiment 3 includes an electronic shutter that switches between storing signal charge and not storing signal charge and four storage sections 4a, 4b, 4c, and 4d. The light-emitting element 1a emits the first light pulse 8a of the wavelength λ₁, and the photoelectric conversion section 3 photoelectrically converts the light 19a returning from the target part 6. After that, the storage section 4a is selected in accordance with control signals 16a, 16b, 16c, 16d, and 16e from the control circuit 7, and the first signal charge 18a is accumulated for a period of time T_{S1} of, for example, 11 to 22 ns. After the period of time T_{S1} has elapsed, a drain 12 is selected in accordance with the control signals 16a, 16b, 16c, 16d, and 16e from the control circuit 7, and electric charge from the photoelectric conversion section 3 is released.

Similarly, the photoelectric conversion section 3 photoelectrically converts the component of the light 11 included in the falling period 13 of the light 19b corresponding to the second light pulse 8b of the wavelength X1 and returning from the target part 6. After that, another storage section 4b is selected in accordance with the control signals 16a, 16b, 16c, 16d, and 16e, and the second signal charge 18b is accumulated for a period of time T_{S2} of, for example, 11 to 22 ns. After the period of time T_{S2} has elapsed, the drain 12 is selected in accordance with the control signals 16a, 16b, 16c, 16d, and 16e from the control circuit 7, and electric charge from the photoelectric conversion section 3 is released.

After this, the light-emitting element 1a is replaced by the light-emitting element 1b, which similarly emits the first and second light pulses 8c and 8d of the wavelength λ₂ in sequence. The storage section 4c corresponds to the first light pulse 8c, and the storage section 4d corresponds to the second light pulse 8d.

Therefore, the component of the light 19a corresponding to a repetitive pulse train of the first light pulses 8a of the wavelength λ₁ and returning from the target part 6 is accumulated as one frame of the first signal charge 18a in the storage section 4a by the photoelectric conversion. After the end of one frame, the first signal charge 18a is outputted as a first electric signal 15a to the control circuit 7. The first electric signal 15a includes the information on scalp blood flow of the wavelength λ₁.

The component of the reflected scattered light 11, which is included in the falling period 13 of the light 19b corresponding to a repetitive pulse train of the second light pulses 8b of the wavelength X1 and returning from the target part 6, is accumulated as one frame of the second signal charge 18b in the storage section 4b by the photoelectric conversion. After the end of one frame, the second signal charge 18b is outputted as a second electric signal 15b to the control circuit 7. The second electric signal 15b includes the information on scalp blood flow of the wavelength λ₁ as well as the information on brain blood flow of the wavelength λ₁.

A component of light 19c corresponding to a repetitive pulse train of the first light pulses 8c of the wavelength λ₂ and returning from the target part 6 is accumulated as one frame of a third signal charge 18c in the storage section 4c by the photoelectric conversion. After the end of one frame, the third signal charge 18c is outputted as a third electric signal 15c to the control circuit 7. The third electric signal 15c includes the information on scalp blood flow of the wavelength λ₂.

The component of the reflected scattered light 11, which is included in the falling period 13 of light 19d corresponding to a repetitive pulse train of the second light pulses 8d of the wavelength λ₂ and returning from the target part 6, is accumulated as one frame of a fourth signal charge 18d in the storage section 4d by the photoelectric conversion. After the end of one frame, the fourth signal charge 18d is outputted as a fourth electric signal 15d to the control circuit 7. The fourth electric signal 15d includes the information on scalp blood flow of the wavelength λ₂ as well as the information on brain blood flow of the wavelength λ₂.

From the four pieces of image information thus acquired, an image of two two-dimensional concentration distributions of oxyhemoglobin and deoxyhemoglobin can be generated as an image that indicates changes in brain blood flow.

Next, biological measuring devices according to modifications of Embodiment 3 of the present disclosure are described.

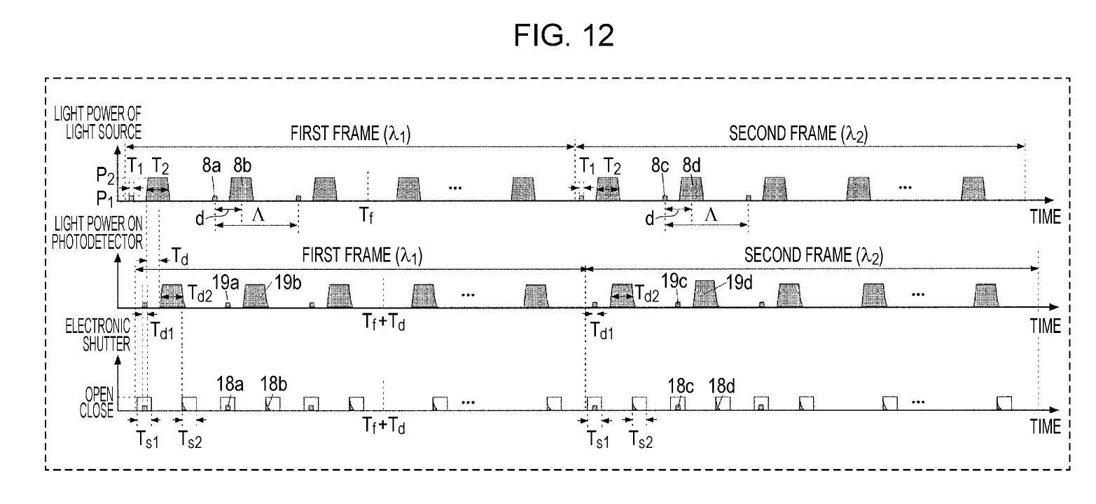

FIG. 12 is a diagram schematically showing a time distribution (upper row) of first and second light pulses, a time distribution (middle row) of a light power on the photodetector 2 in a case where the first and second light pulses are emitted, and the timing and charge storage (lower row) of the electronic shutter according to Modification 1 of Embodiment 3 of the present disclosure.

In this example, two storage sections are used. In the first half of a first frame period, first and second light pulses 8a and 8b having a first wavelength λ₁ are repeatedly emitted, and in the second half of the first frame period, a second light pulse 8b having the first wavelength λ₁ is repeatedly emitted. In the first half of a second frame period, first and second light pulses 8c and 8d both having a second wavelength λ2 are repeatedly emitted, and in the second half of the second frame period, a second light pulse 8d having the second wavelength λ2 is repeatedly emitted.

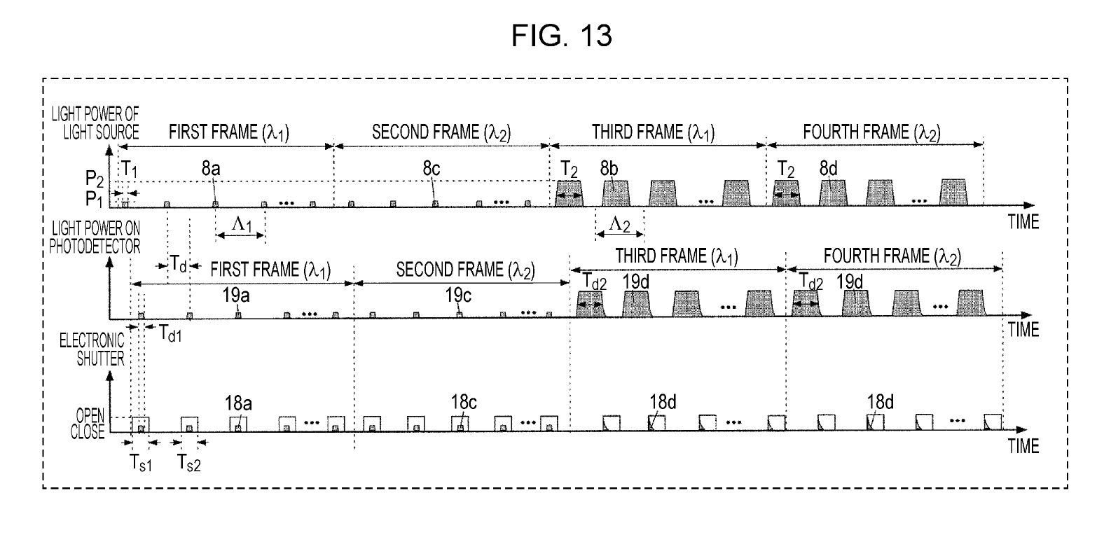

FIG. 13 is a diagram schematically showing a time distribution (upper row) of first and second light pulses, a time distribution (middle row) of a light power on the photodetector 2 in a case where the first and second light pulses are emitted, and the timing and charge storage (lower row) of the electronic shutter according to Modification 2 of Embodiment 3 of the present disclosure.

In this example, one storage section is used. During a first frame period, a first light pulse 8a having a first wavelength λ₁ is repeatedly emitted. During a second frame period, a second light pulse 8c having a second wavelength λ₂ is repeatedly emitted. During a third frame period, a second light pulse 8b having the first wavelength λ₁ is repeatedly emitted. During a fourth frame period, a second light pulse 8d having the second wavelength λ₂ is repeatedly emitted. The photodetector 2 needs only include one storage section. This eliminates the need to switch between storage sections, thus making the configuration simple.

In the foregoing, the biological measuring devices according to Embodiments 1 to 3 have been described. However, the present disclosure is not limited to these embodiments. A biological measuring device based on a combination of the configurations of the biological measuring devices according to the respective embodiments is also encompassed in the present disclosure and can bring about similar effects.

What is claimed is:

1. A measuring device comprising:
a light source that emits at least one first light pulse and at least one second light pulse toward a target part of an object, the at least one second light pulse being different in light power from the at least one first light pulse;
a photodetector that detects at least one first reflected light pulse returning from the target part and at least one second reflected light pulse returning from the target part; and
a control circuit that controls the light source and the photodetector, wherein:
the control circuit causes the light source to emit the at least one first light pulse and the at least one second light pulse at different timings,
the control circuit causes the photodetector to detect a first component and output a first electric signal representing the first component, the first component being a component of light included in the at least one first reflected light pulse, and
the control circuit causes the photodetector to detect a second component and output a second electric signal representing the second component, the second component being a component of light included in the at least one second reflected light pulse during a falling period, the falling period being a period from a point in time at which the at least one second reflected light pulse starts decreasing in light power to a point in time at which the at least one second reflected light pulse finishes decreasing in light power.

2. The measuring device according to claim 1, wherein:
the object is a living body, and
the measuring device further comprises a signal processing circuit that generates blood flow information on the target part through computation based on the first electric signal and the second electric signal.

3. The measuring device according to claim 2, wherein:
the first electric signal includes blood flow information on a surface of the target part,
the second electric signal includes blood flow information on the surface and an interior of the target part, and
the signal processing circuit generates blood flow information on the interior of the target part.

4. The measuring device according to claim 2, wherein:
the photodetector is an image sensor including photodetection cells arrayed two-dimensionally, and
each of the photodetection cells accumulates the first component as a first signal charge,
accumulates the second component as a second signal charge,
outputs, as the first electric signal, an electric signal representing a total amount of the first signal charge, and
outputs, as the second electric signal, an electric signal representing a total amount of the second signal charge.

5. The measuring device according to claim 4, wherein:
the control circuit causes the image sensor to output
a first image signal representing a first two-dimensional distribution of the total amount of the first signal charge accumulated in each of the photodetection cells during a first period,
a second image signal representing a second two-dimensional distribution of the total amount of the second signal charge accumulated in each of the photodetection cells during a second period that is identical to or different from the first period,
a third image signal representing a third two-dimensional distribution of the total amount of the first signal charge accumulated in each of the photodetection cells during a third period preceding the first period, and
a fourth image signal representing a fourth two-dimensional distribution of the total amount of the second signal charge accumulated in each of the photodetection cells during a fourth period preceding the second period, and
the signal processing circuit
receives the first to fourth image signals from the image sensor,
generates a first difference image representing a difference between the first image signal and the third image signal, and
generates a second difference image representing a difference between the second image signal and the fourth image signal.

6. The measuring device according to claim 5, wherein 0.1≤M₁/M₂≤10 is satisfied when
the first difference image includes first pixels each of which has a pixel value exceeding a first threshold, the first pixels forming a first region,
the second difference image includes second pixels each of which has a pixel value exceeding a second threshold, the second pixels forming a second region,
M₁ is an average pixel value of first pixels included in a part of the first region that overlaps the second region, and
M₂ is an average pixel value of second pixels included in a part of the second region that overlaps the first region.

7. The measuring device according to claim 4, wherein the at least one first light pulse has a pulse width that is shorter than a length of time that the photodetector accumulates the first signal charge.

8. The measuring device according to claim 4, wherein the at least one first light pulse has a pulse width that is longer than a length of time that the photodetector accumulates the first signal charge.

9. The measuring device according to claim 4, wherein:
the at least one first light pulse comprises a plurality of first light pulses,
the at least one second light pulse comprises a plurality of second light pulses,
the control circuit causes the light source to repeatedly emit the plurality of first light pulses during a first frame period,
the control circuit causes the photodetector to accumulate the first signal charge in synchronization with emission of each of the plurality of first light pulses,
the control circuit causes the light source to repeatedly emit the plurality of second light pulses during a second frame period that follows the first frame period, and
the control circuit causes the photodetector to accumulate the second signal charge in synchronization with emission of each of the plurality of second light pulses.

10. The measuring device according to claim 1, wherein:
the at least one first light pulse comprises a plurality of first light pulses,
the at least one second light pulse comprises a plurality of second light pulses,
the control circuit causes the light source to alternately emit each of the plurality of first light pulses and each of the plurality of second light pulses, and
a time interval from a center of each of the plurality of first light pulses to a center of a second light pulse that is emitted immediately after each of the plurality of first light pulses is shorter than a time interval from a center of each of the plurality of second light pulses to a center of a first light pulse that is emitted immediately after each of the plurality of second light pulses.

11. The measuring device according to claim 1, wherein either of the at least one first light pulse and the at least one second light pulse has a wavelength of not shorter than 650 nm and shorter than 805 nm and the other of the at least one first light pulse and the at least one second light pulse has a wavelength of longer than 805 nm and not longer than 950 nm.

12. The measuring device according to claim 1, wherein the at least one second light pulse is higher in light power than the at least one first light pulse.

13. A measuring device comprising:
a light source that emits first light pulses and second light pulses toward a target part of an object;
a photodetector that detects first reflected light pulses returning from the target part and second reflected light pulses returning from the target part; and
a control circuit that controls the light source and the photodetector, wherein
light power of each of the second light pulses is higher than light power of each of the first light pulses,
the control circuit causes the light source to alternately emit each of the first light pulses and each of the second light pulses,
the control circuit causes the photodetector to detect a component of light included in the first reflected light pulses, and
the control circuit causes the photodetector to detect a component of light included in the second reflected light pulses.
